(19) United States
(12) Patent Application Publication
(10) Pub. No.: US 2009/0034687 A1
(43) Pub. Date: Feb. 5, 2009

(54) INTRAORAL DENTAL IMAGE SENSOR AND RADIOLOGICAL SYSTEM USING THIS SENSOR

(75) Inventor: Michel Ayraud, Voreppe (FR)

Correspondence Address: LOWE HAUPTMAN & BERNER, LLP, 1700 DIAGONAL ROAD, SUITE 300, ALEXANDRIA, VA, 22314, US

(73) Assignee: E2V Semiconductors, Saint-Egreve (FR)

(21) Appl. No.: 11/909,953

(22) PCT Filed: Jan. 20, 2006
(86) PCT No.: PCT/EP2006/050329

§ 371 Date: Apr. 9, 2008

(30) Foreign Application Priority Data
Apr. 1, 2005 (FR) 0503182

(51) Int. Cl. A61B 6/14

(52) U.S. Cl. 378/168

(57) ABSTRACT

The invention relates to intraoral radiological dental image sensors, i.e. ones placed in a patient's mouth, an X-ray source being placed outside the patient's cheek in order to emit X-rays in the direction of the sensor. According to the invention, the sensor comprises a light source which is modulated by the collected radiological image information, in order to transmit the information optically. The light source, placed inside the patient's mouth, is preferably placed facing an optical fiber which emerges from the mouth. A light diffuser, placed at the end of the fiber, makes it possible to emit nondirectionally. A receiver collects the information and transmits it to an image processing device, in particular with a view to storing the image.

The invention relates to intraoral radiological dental image sensors, i.e. ones placed in a patient's mouth, an X-ray source being placed outside the patient's cheek in order to emit X-rays in the direction of the sensor.

In order to transmit the image information coming from the sensor placed inside the mouth, it is conventional to employ a wire connection which is also used for controlling the sensor and supplying it with energy.

The drawback of a wire connection is that it is fragile (risk of tearing), uncomfortable for the patient if they accidentally pull on the wire, and cumbersome in general installation. In a medical environment, furthermore, the requirements for electrical isolation between the patient and the surrounding electricity supplies are great, and connecting an apparatus (microcomputer) supplied by the power mains to a module which is in the patient's mouth is incompatible with these requirements.

Attempts are therefore made to produce wireless connections both for supplying the energy and for transferring the information coming from the sensor or going to the sensor. Since wireless energy supply (typically: by inductive transmission) is inconvenient, it is generally preferable to use a battery (rechargeable or not) in the sensor placed in the mouth. The image information coming from the sensor must furthermore be transmitted at a high rate (of the order of 20 megabits per second), which is why there is a tendency toward radiofrequency transmission, preferably in the free frequency bands which are in practice those used for wireless computer communication in a local area network (frequencies allocated to WLAN networks: 2.45 GHz for example).

The difficulty, however, is that this radio transmission then risks being interfered with strongly by the presence of other radio transmitters, which are being used more and more frequently in computer environments; peripherals and computer cards of the “WiFi” or “Bluetooth” type may in particular interfere strongly with transmission of the image data from the sensor to the system for processing the image.

This drawback may be overcome by sending the messages redundantly, in order to ensure complete and reliable transmission of the entire image, but this is time-consuming when there is already a large volume of information to be transmitted (typically: several tens of megabits per image).

An “intelligent” transmitter may also be used, which scans those frequencies which are not being used locally in the environment and adapts its own frequency and/or its own data rate as a function of this environment. Such a transmitter necessarily comprises a receiver. The complex electronics for reception, analysis and intelligent processing which this entails make it very difficult to fit everything in the patient's mouth. The encumbrance and power consumption are prohibitive. It is then necessary to divide the system into a sensor located inside the mouth, a connecting wire which starts from the sensor and emerges outside the mouth, and transmitter-receiver in one of the patient's pockets, and an intelligent radiofrequency link between this extraoral transmitter-receiver and the operating system (microcomputer) which has to collect the images. Such a setup is complex.

In order to avoid these drawbacks, the invention is based on the observation that although the sensor is located inside the mouth, it nevertheless proves possible to transmit the image information to the system without using a wire to connect the sensor to the operating system and without using radiofrequency transmission.

The invention provides an intraoral dental radiology system comprising a radiological image sensor adapted to be inserted into the patient's mouth and comprising on the one hand an image detection matrix delivering electrical signals representing a radiological image and on the other hand a light source receiving the signals coming from the matrix and adapted to emit binary light pulses corresponding to a digital image to be transmitted optically, and a light receiver placed at a distance from the patient, adapted to detect a light modulation caused by the light source placed in the mouth and adapted to transmit a signal corresponding to this modulation to an image processing device (particularly in order to store the collected image).

Unexpectedly, therefore, it is proposed to transmit the information by a light source even though the source of the information to be transmitted is in an environment (inside the mouth) which might be expected to preclude transmission by means of light.

In a first variant, which has the advantage of simplicity, the light source emits only inside the patient's mouth but at wavelength and with a power such that a fraction of the emitted light can pass through the cheeks; this fraction of light is superimposed on the ambient light in the form of a modulation of this ambient light, so that the receiver can detect the modulation of the light and therefore the image information transmitted by means of this modulation. Preferably, the light source is a laser diode or a light-emitting diode emitting substantially monochromatic light and the receiver is provided with an optical filter of narrow passband centered around the wavelength of the monochromatic light. The wavelength used is in the band from 700 to 900 nanometers, preferably from 780 to 880 nanometers (red or near infrared), in which band the skin of the cheeks allows a significant fraction of the emitted light to pass through.

A solution which is very advantageous in terms of transmission, because it is not affected by an optical transmission factor of the cheeks, consists in placing a first end of an optical fiber inside the mouth, facing the light source (here again preferably a substantially monochromatic laser diode or light-emitting diode), the optical fiber having a second end and a length such that the second end can emerge from the mouth when the sensor is in the mouth. The second end is provided with a simple light diffuser, which spreads the light received from the fiber into the environment of the patient. The receiver, placed so as to remotely receive a fraction of the light coming from the diffuser, detects the modulation of the light that it receives, which modulation transports the radiological image information. The receiver here again preferably provided with an optical filter centered on the color emitted by the emitter.

The diffuser is preferably a ball of translucent material placed at the end of the optical fiber, so that the light is emitted almost omnidirectionally.

There is preferably a second optical fiber, used to receive information or instructions coming from the operating system and intended for the sensor. This fiber has a first end facing a light receiver integral with the sensor which is located in the mouth and a second end, outside the mouth, connected to a light diffuser which may be the same as that of the first fiber or a different one.

The invention relates not only to the radiological system but also to the intraoral radiological sensor itself, comprising means for converting a radiological image into digital electronic signals and a light source modulated by these electronic signals, making it possible to optically transmit information about the radiological image collected by the sensor, intended for a light receiver located outside the mouth.

Other characteristics and advantages of the invention will become apparent on reading the following detailed description, which is provided with reference to the appended drawings in which:

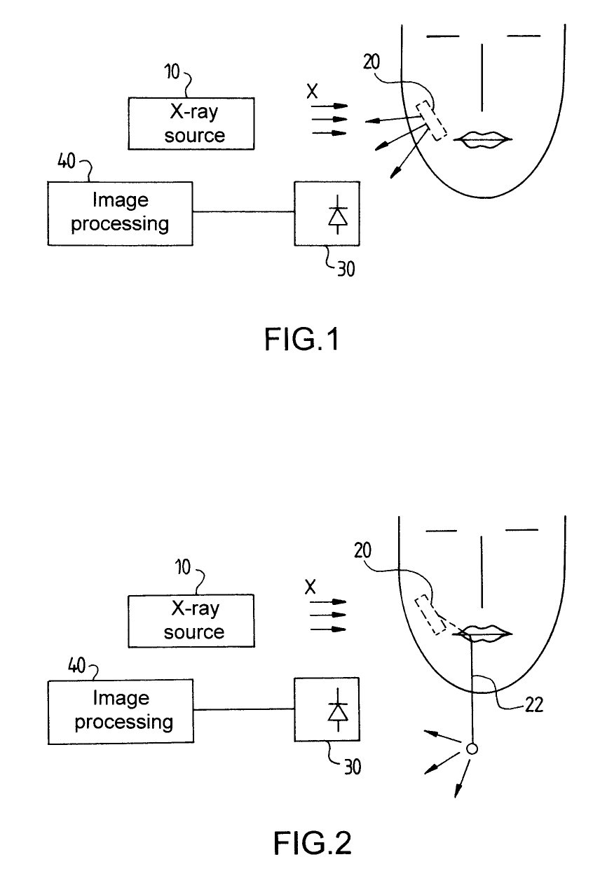

FIG. 1 represents a dental radiology system according to the invention;

FIG. 2 represents a view of the radiological system in a variant of the invention;

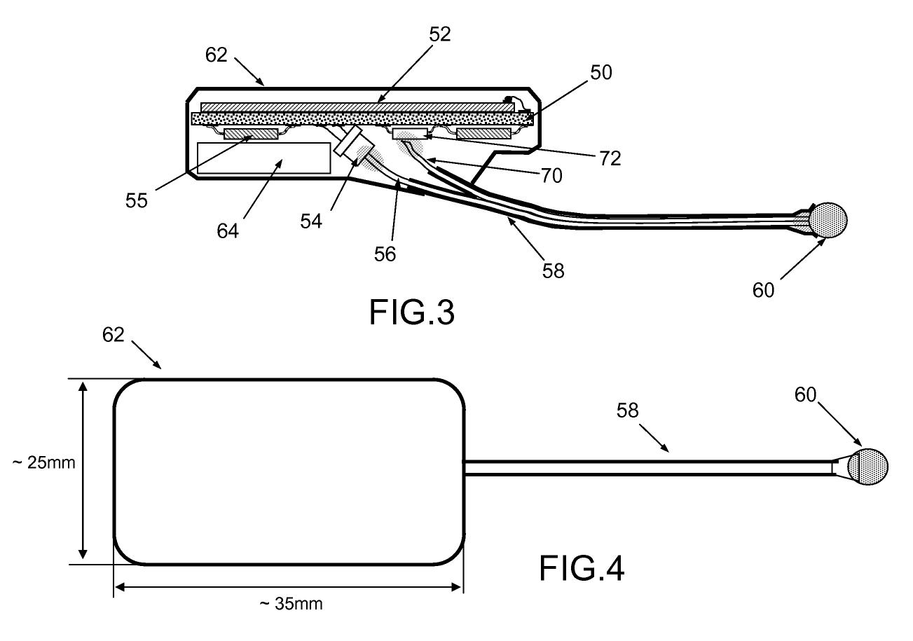

FIG. 3 represents a view in section of a practical embodiment of the image sensor;

FIG. 4 represents a view of the sensor from above.

FIG. 1 schematically represents the dental radiology system according to the invention: it comprises an X-ray source 10 capable of emitting an X-ray flash to the jaw part to be examined, a radiological image sensor 20 placed in the patient's mouth and represented by dashes, a photosensitive receiver 30 placed outside the patient's mouth but not connected electrically to the sensor, and an image analysis system 40 connected to the receiver and capable of receiving digital light signals detected by the receiver, demodulating them in order to extract therefrom image information which they transport, and converting the demodulated signals into an electronic image. This image is intended to be stored in a memory of the system or displayed on a display screen (not shown).

The radiological image sensor conventionally comprises a scintillator layer, which is sensitive to X-rays and converts the received X-ray image into a light image, and a matrix for light image detection placed behind the scintillator layer. The matrix delivers digital electronic signals (or electronic signals that are analog but subsequently converted to digital) representing the radiological image detected at the moment of the X-ray flash.

Unconventionally, the image sensor is furthermore provided with a light emission source (preferably a light-emitting diode) and means for modulating this light source. The modulation means receive the digital electronic signals coming from the matrix and representing the radiological image to be transmitted, and modulate the light emitted by the source as a function of these digital signals.

The light source in the case of FIG. 1 emits inside the mouth; the wavelength of the source is chosen to be in the red or near infrared range (wavelengths for which the patient's flesh (cheeks in particular) has a certain transparency). A modulated light power fraction emerges from the patient's skin, and/or the mouth if it is open. This light power is emitted nondirectionally. The photosensitive receiver collects a part of this power fraction, converts it into electronic signals and transmits it to the image analysis means. The receiver is preferably provided with a wavelength selection filter centered on the main wavelength emitted by the light source, so that at the light sources present in the environment interfere as little as possible with the reception. The passband of the filter should be of the order of 30 nanometers. The receiver may be placed fairly close to the patient, for example a few tens of centimeters or less away. Specifically, it may be carried by a part of the system which also carries the X-ray source. This is because it is known that the X-ray source is generally placed on an articulated arm which allows it to approach within a few centimeters of the cheek. The receiver may be carried by the articulated arm itself.

In a preferred variant represented in FIG. 2, the difficulties of optical transmission through the patient's cheeks are obviated and an optical fiber 22 is provided, a first end of which is fixed facing the light source (inside the patient's mouth) and another end of which emerges outside the mouth. This other end diffuses so that the light collected by the fiber from the source is emitted outside nondirectionally, or in any event with weak directionality. A translucent ball may be adhesively bonded to this end in order to fulfill this function of a light diffuser. The ball may be made of alumina or plastic, such as white nylon. It may be a ball with a diameter of about 1 mm protected by a shell of overmolded plastic, which is transparent at the wavelength in question.

A fraction of the emitted light is collected remotely by the receiver and transmitted to the image analysis system 40.

Details of an intraoral sensor according to the invention can be seen in FIG. 3, in an embodiment with optical fibers emerging from the patient's mouth.

The sensor comprises a printed circuit 50 carrying on one face an integrated circuit chip 52, on one face of which the radiological image sensor per se is formed, namely a light image detection matrix covered with a scintillator reacting to the X-rays. The matrix establishes electronic signals representing the light levels of the pixels; the chip 52, with a side length of a few centimeters, comprises of the analog-digital conversion circuits making it possible to convert the electronic signal into digital signals representing each pixel.

The signals are applied to a light-emitting diode (LED) 54 or a laser diode, mounted on the printed circuit 50, preferably on its other face. The diode emits light pulses according to the digital signals which it receives from the chip 52. The emitted light is preferably infrared light with a wavelength which is nonhazardous for the eyes and preferably quite strongly monochromatic. The printed circuit 52 may comprise circuitry elements other than the component 55 represented in FIG. 3.

An optical fiber 56, contained in a flexible sheath of plastic material 58 about 10 cm long, has an end adhesively bonded (by a transparent optical adhesive) in front of the emission face of the diode 54. This fiber has another end adhesively bonded by a transparent optical adhesive to a light diffuser 60. The diffuser 60 is a ball of translucent material (plastic material such as white nylon or alumina). Its substantially spherical shape allows the light coming from the optical fiber to be distributed in a wide solid angle, so that there is no need to find a specific orientation of the optical fiber in order for the receiver 30 (FIG. 2) to receive the emitted light. The sheathed optical fiber, since it is not attached to anything at its second end, does not risk being torn by an untimely movement. The diameter of the sheathed fiber is about 1 mm in diameter.

All of the printed circuit 50 and its components, with the integrated circuit chip 52, the diode 54 and the first end of the sheathed optical fiber, are housed in a leaktight casing 62 from which the optical fiber emerges. The casing preferably also contains a cell or a battery 64 allowing independent electricity supply of the printed circuit and the component which it carries. Provision may also be made for the casing to be made of transparent plastic material and for the sheathed optical fiber to be adhesively bonded on the casing, facing the laser diode 54, instead of penetrating into the casing.

In an exemplary embodiment, the dimensions of the casing of the sensor are as follows: L=approximately 35 mm, 1=approximately 25 mm, H=approximately 10 mm.

Optionally, provision may be made for the radiological system also to comprise means for optical communication in the opposite direction, i.e. from the system to the sensor placed in the mouth. This option is provided in the sensor of FIG. 3 in the form of a second optical fiber 70 and a photodiode 72. The photodiode is mounted on the printed circuit 50. The second optical fiber is preferably mounted in the same sheath as the first, and it has a first end adhesively bonded with an optical adhesive onto the same diffusion ball 60 so as to collect light arriving substantially in any direction. The photodiode can receive information or instructions coming from the system, and have them processed (in particular by the integrated circuit chip 52). A light emitter associated with the system may emit light pulses conveying this information or these instructions, to which end the wavelength of the emitted light is preferably different than the wavelength emitted by the laser diode 54, so that there is no perturbing interference between the outgoing light pulses and the incoming light pulses.

By way of example, the incoming pulses (from the system to the sensor) may be used to send information for triggering an X-ray flash, or to request the sensor to send or resend an image or a part of an image, or to parameterize certain functions of the sensor (exposure time, etc.). The information rate in the incoming direction may be much less than in the outgoing direction, since there is no image to be transmitted.

It will be noted that a bidirectional system can be provided even with a single optical fiber, provided that there are splitting means at the output of the fiber in order to transmit the received information to a light receiver placed on the sensor while separating it from the emitted information; the splitting may be carried out for example with the aid of a dichroic mirror; it may also be carried out with a conventional mirror, with temporal separation by using a protocol which periodically extinguishes the internal light source during times reserved for receiving information coming from outside the patient's mouth. The laser diode itself may be used as the photodiode, if it is not being used per se during transmission.

1. An intraoral dental radiology system comprising a radiological image sensor adapted to be inserted into the patient's mouth and comprising on the one hand an image detection matrix delivering electrical signals representing a radiological image and on the other hand a light source receiving the signals coming from the matrix and adapted to emit binary light pulses corresponding to a digital image to be transmitted optically, and a light receiver placed at a distance from the patient, adapted to detect a light modulation caused by the light source placed in the mouth and adapted to transmit a signal corresponding to this modulation to an image processing device.

2. The system as claimed in claim 1, wherein the light source is placed on the sensor and emits light inside the patient's mouth when the sensor is placed inside the mouth, at a wavelength and with a power such that a fraction of the emitted light can pass through the cheeks.

3. The system as claimed in claim 2, wherein the light source is a laser diode or a light-emitting diode emitting substantially monochromatic light and the receiver is provided with an optical filter of narrow passband centered around the wavelength of the monochromatic light.

4. The system as claimed in claim 3, wherein the wavelength used is in the band from 780 to 880 nanometers (red or near infrared), in which band the skin of the cheeks allows a significant fraction of the emitted light to pass through.

5. The system as claimed in claim 1, wherein the light source is connected to a first end of an optical fiber inside the mouth, the optical fiber having a second end and a length such that the second end can emerge from the mouth when the sensor is in the mouth.

6. The system as claimed in claim 1, wherein the second end of the fiber is provided with a simple light diffuser which spreads the light received from the fiber into the environment of the patient.

7. The system as claimed in claim 6, wherein the diffuser is a ball of translucent material placed at the end of the optical fiber.

8. The system as claimed in claim 5, wherein the sensor comprises means for receiving information or instructions coming from the operating system and intended for the sensor, this information being received in optical form by the optical fiber and transmitted to a light receiver, splitting means being provided at the first end of the optical fiber in order to separate the emitted information from the received information and to transmit the received information to the light receiver.

9. The system as claimed in claim 6, wherein it comprises a second optical fiber used to receive information or instructions coming from the operating system and intended for the sensor, and in that the sensor comprises a light receiver facing one end of the second optical fiber, the optical fiber having a length such that the other end can emerge from the mouth when the sensor is in the mouth.

10. The system as claimed in claim 9, wherein the second end of the second optical fiber is connected to a light diffuser.

11. An intraoral radiological sensor, comprising means for converting a radiological image into digital electronic signals and a light source modulated by these electronic signals, making it possible to optically transmit information about the radiological image collected by the sensor, intended for a light receiver located outside the mouth, and an optical fiber having a first end placed facing the light source and having a length such that a second end can emerge from a patient's mouth when the sensor is in the mouth.

12. (canceled)

13. The sensor as claimed in claim 11, wherein a light diffuser is placed at the second end of the fiber.

14. The system as claimed in claim 6, wherein the sensor comprises means for receiving information or instructions coming from the operating system and intended for the sensor, this information being received in optical form by the optical fiber and transmitted to a light receiver, splitting means being provided at the first end of the optical fiber in order to separate the emitted information from the received information and to transmit the received information to the light receiver.

15. The system as claimed in claim 7, wherein the sensor comprises means for receiving information or instructions coming from the operating system and intended for the sensor, this information being received in optical form by the optical fiber and transmitted to a light receiver, splitting means being provided at the first end of the optical fiber in order to separate the emitted information from the received information and to transmit the received information to the light receiver.

16. The system as claimed in claim 7, wherein it comprises a second optical fiber used to receive information or instructions coming from the operating system and intended for the sensor, and in that the sensor comprises a light receiver facing one end of the second optical fiber, the optical fiber having a length such that the other end can emerge from the mouth when the sensor is in the mouth.
